(19) United States
(12) Patent Application Publication
(10) Pub. No.: US 2024/0299567 A1
(43) Pub. Date: Sep. 12, 2024

(54) RECOMBINANT POLYPEPTIDES, CONJUGATES COMPRISING THE SAME, AND USES THEREOF

(71) Applicants: Ming-Che SHIH, Culver City, CA (US); Academia Sinica, Taipei City (TW)

(72) Inventors: Che MA, New Taipei City (TW); Wen-Hwa LEE, Taipei City (TW); Tracer YONG, Tainan City (MY)

(73) Assignee: Academia Sinica, Taipei City (TW)

(21) Appl. No.: 18/272,107

(22) PCT Filed: Jan. 14, 2022
(86) PCT No.: PCT/US2022/012414

§ 371 Date: Jul. 13, 2023

Related U.S. Application Data
(60) Provisional application No. 63/137,709, filed on Jan. 14, 2021.

(51) Int. Cl. A61K 47/64; A61K 39/00; A61P 35/04; C07K 14/715

(52) U.S. Cl. CPC A61K 47/646; A61K 47/6415; A61P 35/04; C07K 14/7155; A61K 39/00; A61K 2039/575; A61K 2039/6031; A61K 2039/645; C07K 2319/40

(57) ABSTRACT

Disclosed herein is a recombinant polypeptide comprising 1 to 20 copies of an IL-17RB inactivation site (IRIS) sequence. Also disclosed herein is the use of the recombinant polypeptide in the preparation of a conjugate for the treatment of cancers.

### BACKGROUND OF THE INVENTION

### 1. Field of the Invention

The present disclosure relates to the treatment of cancers. More specifically, the disclosure invention relates to a conjugate comprising a plurality of interleukin-17 receptor B (IL-17RB) inactivation site (IRIS) sequence, and uses of the conjugate for treating cancers.

### 2. Description of Related Art

In the past, vaccines were used to prevent bacterial or viral infections that harm humans, and have achieved great success, such as the Vaccinia vaccine for preventing Smallpox, the Bacille Calmette-Guerin vaccine for preventing Tuberculosis, the Hepatitis B vaccine for preventing Hepatitis B, the Influenza A vaccine for preventing Influenza A infection, and the like. Recently, a novel category of vaccines called cancer vaccines was developed to induce effective and sustained antitumor immunity, and some of them have demonstrated to be a potential adjuvant therapy for treatments, such as surgery, chemotherapy and radiotherapy, thereby improving the efficacy of these treatments. Nonetheless, a cancer vaccine with a robust ability in eliciting tumor-specific immunity as well as depriving immune resistance at the same time remains a challenge.

One of the key essentials in developing a cancer vaccine is to find out the target antigen expressed on cancer cells. The target antigen of cancer cells had better only appear on cancer cells and not on normal cells, so that the target antigen would be recognized as an exogenous allothigene by immune cells, which in turn triggers the immune system to launch an overall attack against the target antigen, eventually resulting in eliminating the cancer cells having the target antigen expressed thereon. Numerous cancer vaccines were developed in accordance with such the principle.

IL-17RB is a cytokine receptor, which specifically binds to IL-17B and IL-17E (or IL-25), but not IL-17A or IL-17C. The expression level of IL-17RB is reported to be proportional to the proliferation, and invasion ability of cancer cells, such as breast cancer cells, cervical cancer cells, gastric cancer cells, lung cancer cells, pancreatic cancer cells, and thyroid cancer cells. Further, owing that IL-17RB is overexpressed in cancer cells, and blocking the IL-17B/E-IL-17RB signaling pathway would compromise the malignancy of caners, IL-17RB may serve as a therapeutic target for cancer treatment. However, current strategies employing monoclonal antibodies as the therapeutic agents fail to provide a satisfactory result due to the limitation of high cost and short action time of the antibodies.

In view of the foregoing, there exists in the related art a need for a novel method of blocking IL-17RB signalling pathway thereby providing a means to treat cancers.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a recombinant polypeptide, which comprises 1 to 20 copies of an interleukin-17 receptor B (IL-17RB) inactivation site (IRIS) sequence having an amino acid sequence at least 85% identical to SEQ ID NO: 1 or SEQ ID NO: 2, wherein when more than one copy of the IRIS sequence is present in the recombinant polypeptide, each copy of the IRIS sequence is serially connected to each other.

According to one specific embodiment of the present disclosure, in the present recombinant polypeptide, the IRIS sequence has the amino acid sequence 100/6 identical to SEQ ID NO: 1 or SEQ ID NO: 2.

According to one specific embodiment of the present disclosure, the present recombinant polypeptide comprises two copies of the IRIS sequence.

Optionally, the present recombinant polypeptide may be further modified at the N- or C-terminus, or both, in order to prevent unwanted reaction between the recombinant polypeptide and the linker. According to some optional embodiments of the present disclosure, the N-terminus of the present recombinant polypeptide is acetylated, formylated, methylated, carbamylated, pegylated, phosphorylated, or glycosylated. Additionally or alternatively, the C-terminus of the present recombinant polypeptide is amidated, glypiated, biotinylated, or glycosylated.

In another aspect, the present disclosure is directed to a conjugate for use in manufacturing a cancer vaccine; the conjugate comprises,

a carrier protein;

a plurality of the present recombinant polypeptides; and

a plurality of linkers for linking the plurality of the present recombinant polypeptides to the carrier protein;

wherein,

each of the linkers is linked to each of the present recombinant polypeptides at one end of the linker and the carrier protein at the other end of the linker independently through NHS ester amine reaction, thio-succinimide reaction, pyridyldithiol to sulfhydryl reaction, bromoacetyl to sulfhydryl reaction, or iodoacetyl to sulfhydryl reaction.

The carrier protein suitable for use in the present conjugate includes, but is not limited to, edema factor (EF) of Bacillus anthracis, lethal factor (LF) of Bacillus anthracis, bovine serum albumin (BSA), CRM9, CRM45, CRM102, CRM103, CRM107, CRM176, CRM197, CRM228, diphtheria toxoid, heat-labile enterotoxin (LT) of Escherichia coli, heat-stable enterotoxin (ST) of Escherichia coli, human serum albumin, keyhole limpet hemocyanin (KLH), ovalbumin, pertussis toxoid, pneumococcal adhesin protein A (PsaA), pneumococcal surface protein A (PspA), pneumolysin, porins, exotoxin A from Pseudomonas aeruginosa, tuberculin, tetanus toxoid, transferrin binding proteins, and a combination thereof. In one specific embodiment of the present disclosure, the carrier protein is CRM197 (SEQ ID NO: 13).

According to some embodiments of the present disclosure, the linker may be succinimidyl 3-(bromoacetamido) propionate (SBAP), succinimidyl 4-(N-maleimido methyl) cyclohexane-1-carboxylate (SMCC), or N-β-maleimidopropyl-oxysuccinimide ester (BMPS). In one specific example of the present disclosure, the linker is SBAP.

Further, the present recombinant polypeptide of the conjugate may be further modified at the N- and/or C-terminus. According to some optional embodiments of the present disclosure, the N-terminus of the present recombinant polypeptide is acetylated, formylated, methylated, carbamylated, pegylated, phosphorylated, or glycosylated. Additionally or alternatively, the C-terminus of the present recombinant polypeptide is amidated, glypiated, biotinylated, or glycosylated.

Also disclosed herein is an immunogenic composition, which comprises the present conjugate, and a pharmaceutically acceptable carrier. According to some embodiments of the present disclosure, the present immunogenic composition further comprises an adjuvant.

In yet another aspect, the present disclosure is directed to a method for treating a cancer in a subject by using the conjugate or immunogenic composition as described herein. Specifically, the method comprises the step of administering to the subject an effective amount of the present conjugate or immunogenic composition.

Exemplary cancers that are treatable with the present method include, but are not limited to, bladder cancer, biliary cancer, bone cancer, brain tumor, breast cancer, cervical cancer, colorectal cancer, esophageal cancer, epidermal carcinoma, gastric cancer, gastrointestinal stromal tumor (GIST), glioma, hematopoietic tumors of lymphoid lineage, hepatic cancer, Kaposi's sarcoma, leukemia, lung cancer, lymphoma, intestinal cancer, melanoma, myeloid leukemia, pancreatic cancer, prostate cancer, retinoblastoma, ovary cancer, renal cell carcinoma, spleen cancer, squamous cell carcinoma, thyroid cancer, and thyroid follicular cancer. In one specific embodiment of the present disclosure, the subject has the breast cancer.

According to some embodiments of the present disclosure, the cancer may be an in situ cancer or a metastatic cancer. In one specific embodiment, the cancer is a metastatic cancer.

According to some embodiments of the present disclosure, the subject is a human.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a schematic diagram depicting the preparation of the present vaccine according to one example of the present disclosure;

FIG. 2 is the results of matrix-assisted laser desorption/ionization-time of flight mass spectrometry (MALDI-TOF MS) that characterized the preparation of specified products. Top panel: the MALDI-TOF MS signal of Carrier Protein (i.e., CRM197); meddle panel: the MALDI-TOF MS signal of Carrier:XLnkr (i.e., CRM197 activated by the linker SBAP, CRM197-SBAP); bottom panel: the MALDI-TOF MS signal of Vaccine (i.e., the present conjugate, CRM197-SBAP-hIRIS2);

FIGS. 3A-3C depict the efficacy of the present vaccine on inducing immune response against the recombinant human IL-17RB (rhIL-17RB) in vivo. FIG. 3A: mice sera from the Carrier (CRM197) and the Vaccine (CRM197-SBAP-hIRIS2) groups were obtained on Day 0, 14 and 28 after vaccination, and were examined by enzyme linked immunosorbent assay (ELISA) to characterize their reactivity against rhIL-17RB (especially for its ectodomain, rhIL-17RB.ECD). The half maximal effective concentration (EC50) for the vaccine was further analyzed from the ELISA results and plotted with filled dots. FIG. 3B: the results depicting the EC50 (fold dilution) of antiserum from mice immunized with vaccines CRM197-SBAP-hIRIS1 or CRM197-SBAP-hIRIS2 at specific peptide-protein ratios (PPRs). FIG. 3C: the results depicting the EC50 of IgG subtypes (i.e., IgG1 and IgG2a) in each mouse with higher immune response; and

FIGS. 4A-4F depict the efficacy of the vaccine on tumor growth and metastasis in a mouse syngeneic tumor model. FIG. 4A: the ELISA results depicting the immunogenicity yielded in control Balb/c mice (n=5) following the therapeutic vaccination scheme as described in “Materials and Methods”, in which the control mice were given the vaccine CRM197-SBAP-hIRIS2 and were not orthotopically transplanted with tumors. The IgG responses specific to IL-17RB are presented in line and the EC50 is presented as dots. FIG. 4B: tumor growth in the mice in the PBS, the Carrier control (CRM197), and the Vaccine (CRM197-SBAP-hIRIS2) treatment groups. T-tests were carried out between the PBS and the Vaccine treatment groups, and the p values are as indicated. FIG. 4C: tumor weight of the primary tumors in the mice in the indicated treatment groups at the endpoint of the experiment. FIG. 4D: luminescence signal intensity of the primary tumors in the mice in the indicated treatment groups on Day 13 of the treatment. FIG. 4E: luminescence signal intensity of the tumors metastasized in the lungs in the mice in the indicated treatment groups. FIG. 4F: H-score on the immunohistochemical (IHC) staining of pERK1/2 in the tumors in the mice in the indicated treatment groups. Non-significant (NS): p>0.05; *: p<0.05.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a,” “an,” and “the” include the plural reference unless the context clearly dictates otherwise. Also, as used herein and in the claims, the terms “at least one” and “one or more” have the same meaning and include one, two, three, or more. The practice of the present invention will employ, unless otherwise indicated, conventional techniques of molecular biology, synthetic chemistry, structural biology, and immunology, which are within the skill of the art. Such techniques are explained fully in the literature.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “linker” as used herein refers to any chemical moiety that is capable of linking the present recombinant polypeptide or a cap protein, to a carrier protein (e.g., CRM197), in a stable, covalent manner. Linkers can be susceptible to or be substantially resistant to acid-induced cleavage, light-induced cleavage, peptidase-induced cleavage, esterase-induced cleavage, and disulfide bond cleavage, at conditions under which the present recombinant polypeptide, the cap protein, or the carrier protein remains active. Suitable linkers are well known in the art, preferably, linkers bind to an amine group or a sulfhydryl group on the present recombinant polypeptide, the cap protein, or the carrier protein. Therefore, linkers preferably contain functional groups which can form a binding with the amine group or the sulfhydryl group by targeting them.

The term “conjugate” as used herein refers to a carrier protein (e.g., CRM197) that is conjugated with the present recombinant polypeptide and/or a cap protein, and has the structure of formula (I):

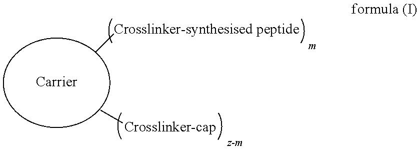

wherein z represents the number of the linkers conjugated to the carrier protein, and m represents the number of the linkers further conjugated with the recombinant polypeptide; z and m are independently an integer between 1 to 100, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. First of all, the number of the linkers conjugated with the carrier protein (i.e., the z number) may vary with the types of the carrier protein, which has a certain amount of active sites on its surface that can conjugate with the linkers, as well as the condition for carrying out the process of the conjugation. For example, the carrier protein CRM197 theoretically has about 40 active sites for conjugating with the linkers, and may in fact only has about 20 or 25 linkers conjugated thereon (i.e., z=20 or 25). In the case when 20 linkers are conjugated to the carrier protein (i.e., z=20), and among them, about 17 linkers are further conjugated with the present recombinant polypeptide (i.e., m=17), then, there may be about 3 remaining linkers for further conjugation with the cap protein (i.e., z-m=3). In the case when 25 linkers are conjugated on the surface of the carrier protein (i.e., z=25), and among them, about 17 linkers are further conjugated with the present recombinant polypeptide (i.e., m=17); then, there may be about 8 remaining linkers for further conjugation with the cap protein (i.e., z-m=8).

The terms “treatment” and “treating” as used herein may refer to a curative or palliative measure. In particular, the term “treating” as used herein refers to the application of the present recombinant polypeptide, conjugate, immunogenic composition, and method to a subject, who has a cancer, a symptom associated with a cancer, a disease or disorder secondary to a cancer, with the purpose to partially or completely alleviate, ameliorate, relieve, delay onset of, inhibit progression of, reduce severity of, and/or reduce incidence of one or more symptoms or features of a cancer.

The terms “cancer” and “tumor” are used alternatively in the present disclosure, and preferably refer to or describe the physiological condition in mammals that is typically characterized by unregulated cell growth. Cancers in this respect include in situ cancers, metastatic cancers, and/or drug-resistant cancers.

The term “subject” or “patient” refers to an animal including the human species that is treatable with the recombinant polypeptide, conjugate, immunogenic composition, and method of the present disclosure. The term “subject” or “patient” intended to refer to both the male and female gender unless one gender is specifically indicated. Accordingly, the term “subject” or “patient” comprises any mammal which may benefit from treatment of cancer. Examples of a “subject” or “patient” include, but are not limited to, a human, rat, mouse, guinea pig, monkey, pig, goat, cow, horse, dog, cat, bird and fowl. In an exemplary embodiment, the subject is a mouse. In another exemplary embodiment, the subject is a human.

The term “administered,” “administering” or “administration” are used interchangeably herein to refer means administering the recombinant polypeptide, conjugate, immunogenic composition, and method as described in the present disclosure to a subject in need.

The term “an effective amount” as used herein refers to an amount effective, at dosages, and for periods of time necessary, to achieve the desired therapeutically desired result with respect to the treatment of cancers. For example, in the treatment of a cancer, the present recombinant polypeptide, conjugate, immunogenic composition, and method would be effective in preventing the cancer cells from spreading and/or growing. An effective amount of an agent is not required to cure a disease or condition but will provide a treatment for a disease or condition such that the onset of the disease or condition is delayed, hindered or prevented, or the disease or condition symptoms are ameliorated. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the like. Effective amount may be expressed, for example, as the total mass of the active agent (e.g., in grams, milligrams or micrograms) or a ratio of mass of the active agent to body mass, e.g., as milligrams per kilogram (mg/kg). The effective amount may be divided into one, two or more doses in a suitable form to be administered at one, two or more times throughout a designated time period. Preferably, the effective amount refers to human equivalent dose (HED), which is the maximum safe dosage for use in human subjects. HED may be calculated by following the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

### II. Description of the Invention

The present disclosure is based, at least in part, on the discovery that blocking the IL-17RB inactivation site (IRIS) epitope may interrupt IL-17RB signalling, thus prevents the cancer cells from spreading and/or growing. Based on this discovery, the present disclosure aims at providing a novel anti-cancer strategy, in which a cancer vaccine against the IRIS epitope is developed, which may trigger a host's immune responses to block the IRIS epitope and prevent cancers, especially the IRIS-overexpressed cancers, from spreading and/or growing.

### 1. The Recombinant Polypeptide

Accordingly, the first aspect of the present disclosure is directed to a recombinant polypeptide that comprises the IRIS epitope. According to some embodiments of the present disclosure, the IRIS epitope is derived from a mouse, i.e., an mIRIS having an amino acid sequence at least 85% identical to SEQ ID NO: 1, such as 85%, 86%, 87%, 88%, 89%, 90%, 91%, 92%, 93%, 94%, 95%, 96%, 97%, 98%, 99%, or 100% identical to SEQ ID NO: 1. According to certain embodiments of the present disclosure, the IRIS epitope is derived from a human, i.e., an hIRIS having an amino acid sequence at least 85% identical to SEQ ID NO: 2, such as 85%, 86%, 87%, 88%, 89%, 90%, 91%, 92%, 93%, 94%, 95%, 96%, 97%, 98%, 99%, or 100% identical to SEQ ID NO: 2. In one specific example, the mIRIS has the amino acid sequence of SEQ ID NO. 1. In another example, the hIRIS has the amino acid sequence of SEQ ID NO: 2.

Depending on desired purposes, the recombinant polypeptide may comprise 1 to 20 copies (i.e., 1, 2, 3, 4, 5, 6, 7, 8, 9, 10, 11, 12, 13, 14, 15, 16, 17, 18, 19, or 20 copies) of the IRIS sequence (i.e., the mIRIS or the hIRIS). Preferably, the present recombinant polypeptide comprises 1 to 10 copies of the IRIS; more preferably, the present recombinant polypeptide comprises 1 to 5 copies of the IRIS. According to one working example of the present disclosure, the present recombinant polypeptide comprises two copies of the IRIS sequence. In the case when more than one copy of the IRIS is present in the recombinant polypeptide, each copy of the IRIS is serially connected to each other.

In one embodiment of the present disclosure, the present recombinant polypeptide comprises one copy of the mIRIS (i.e., mIRIS1), and has the amino acid sequence of SEQ ID NO: 3. In another embodiment of the present disclosure, the present recombinant polypeptide comprises one copy of the hIRIS (i.e., hIRIS1), and has the amino acid sequence of SEQ ID NO: 4. In another embodiment of the present disclosure, the present recombinant polypeptide comprises two copies of the mIRIS (i.e., mIRIS2), and has the amino acid of SEQ ID NO: 5. In another embodiment of the present disclosure, the present recombinant polypeptide comprises two copies of the hIRIS (i.e., hIRIS2), and has the amino acid sequence of SEQ ID NO: 6. In still another embodiment of the present disclosure, the present recombinant polypeptide comprises five copies of the mIRIS (i.e., mIRIS5), and has the amino acid of SEQ ID NO: 7. In still another embodiment of the present disclosure, the present recombinant polypeptide comprises five copies of the hIRIS (i.e., hIRIS5), and has the amino acid sequence of SEQ ID NO. 8. In still another specific embodiment of the present disclosure, the present recombinant polypeptide comprises ten copies of the mIRIS (i.e., mIRIS10), and has the amino acid of SEQ ID NO: 9. In still another embodiment of the present disclosure, the present recombinant polypeptide comprises ten copies of the hIRIS (i.e., hIRIS10), and has the amino acid sequence of SEQ ID NO: 10. In yet still another specific embodiment of the present disclosure, the present recombinant polypeptide comprises twenty copies of the mIRIS (i.e., mIRIS20), and has the amino acid of SEQ ID NO: 11. In yet still another embodiment of the present disclosure, the present recombinant polypeptide comprises twenty copies of the hIRIS (i.e., hIRIS20), and has the amino acid sequence of SEQ ID NO: 12.

### 2. The Conjugate

Another aspect of the present disclosure pertains to a conjugate for use in manufacturing a cancer vaccine. The conjugate comprises,

a carrier protein;

a plurality of the present recombinant polypeptides; and

a plurality of linkers for linking the plurality of the present recombinant polypeptides to the carrier protein;

wherein,

each of the linkers is linked to each of the present recombinant polypeptides at one end and the carrier protein at the other end via any one of NHS ester amine reaction, thio-succinimide reaction, pyridyldithiol to sulfhydryl reaction, bromoacetyl to sulfhydryl reaction, or iodoacetyl to sulfhydryl reaction.

Depending on desired purposes, the carrier protein may be EF of Bacillus anthracis, LF of Bacillus anthracis, BSA, CRM9, CRM45, CRM102, CRM103, CRM107, CRM176, CRM197, CRM228, diphtheria toxoid, LT of Escherichia coli, ST of Escherichia coli, human serum albumin, KLH, ovalbumin, pertussis toxoid, PsaA, PspA, pneumolysin, porins, exotoxin A from Pseudomonas aeruginosa, tuberculin, tetanus toxoid, or transferrin binding protein. According to one preferred embodiment of the present disclosure, the carrier protein is CRM197, and comprises the amino acid sequence of SEQ ID NO: 13.

According to some embodiments, the present recombinant polypeptide is linked to the carrier protein by a linker having an NHS ester group at one end, and a maleimide group at the other end. In these embodiments, the NHS ester group of the linker is linked to the amine group of the recombinant polypeptide via NHS ester amine reaction, and the maleimide group of the linker is linked to the sulfhydryl group of the carrier protein via thio-succinimide reaction. Examples of the linker having a NHS and a maleimide group include, but are not limited to, N-(α-maleimidoacetoxy)succinimide ester (AMAS), BMPS, N-ε-malemidocaproyl-oxysuccinimide ester (EMCS), N-(γ-maleimidobutyryloxy)succinimide ester (GMBS), N-κ-maleimidoundecanoyl-oxysulfosuccinimide ester (sulfo-KMUS), m-maleimidobenzoyl-N-hydroxysuccinimide ester (MBS), SMCC, succinimidyl 4-(N-maleimidomethyl)cyclohexane-1-carboxy-(6-amidocaproate)) (LC-SMCC), succinimidyl 4-(p-maleimidophenyl)butyrate (SMPB), and succinimidyl 6-((beta-maleimidopropionamido)hexanoate) (SMPH). In one preferred example of the present disclosure, the linker is SMCC. In another preferred example of the present disclosure, the linker is BMPS.

According to some embodiments, the present recombinant polypeptide is linked to the carrier protein by a linker having an NHS ester group at one end, and a bromoacetyl or iodoacetyl at the other end. In these embodiments, the NHS ester group of the linker is linked to the amine group of the recombinant polypeptide via NHS ester amine reaction, and the bromoacetyl or iodoacetyl group of the linker is linked to the sulfhydryl group of the carrier protein via bromoacetyl to sulfhydryl reaction, or iodoacetyl to sulfhydryl reaction. Exemplary linkers are SBAP, succinimidyl iodoacetate (SIA), or succinimidyl (4-iodoacetyl)aminobenzoate (SIAB). In one preferred example of the present disclosure, the linker is SBAP.

According to certain embodiments, the present recombinant polypeptide is linked to the carrier protein by a linker having an NHS ester group at one end, and a pyridyldithiol group at the other end. In these embodiments, the NHS ester group of the linker is react to the amine group of the recombinant polypeptide via NHS ester amine reaction, and the pyridyldithiol group of the linker is reacted to the sulfhydryl group of the carrier protein via pyridyldithiol to sulfhydryl reaction. Such linkers are, for example, 4-succinimidyloxycarbonyl-alpha-methyl-α(2-pyridyldithio)toluene (SMPT), succinimidyl 3-(2-pyridyldithio)propionate (SPDP), or succinimidyl 6-(3(2-pyridyldithio)propionamido)hexanoate (LC-SPDP).

Optionally, the present conjugate further comprises a plurality of cap proteins independently linked to the surface of the carrier protein. Examples of the cap protein suitable to be used in the present disclosure include, but are not limited to, adeno-associated virus (AAV) cap protein, β-actinin, CapG, CapZ, Cap32/34, CARMIL, cytochalasin, Ena/VASP, f-actin-capping protein, formins, gelsolin, human macrophage-capping protein, and myotrophin. The linkage between the cap protein and the carrier protein is similar to that of the recombinant polypeptide and the carrier protein. For example, the cap protein may be linked to the carrier protein via a linker having a NHS ester group at one end and a maleimide group at the other end (e.g., SMCC or BMPS), in which the NHS ester group of the linker is linked to the amine group of the cap protein via NHS ester amine reaction, and the maleimide group of the linker is linked to the sulfhydryl group of the carrier protein via thio-succinimide reaction. Alternatively, the cap protein may be linked to the carrier protein via a linker having a NHS ester group at one end and a bromoacetyl or iodoacetyl group at the other end (e.g., SBAP), in which the NHS ester group of the linker is linked to the amine group of the cap protein via NHS ester amine reaction, and the bromoacetyl or iodoacetyl group of the linker is linked to the sulfhydryl group of the carrier protein via bromoacetyl to sulfhydryl reaction, or iodoacetyl to sulfhydryl reaction. For another example, the cap protein may be linked to the carrier protein via a linker has a NHS ester group at one end and a pyridyldithiol group at the other end (e.g., SMPT), in which the NHS ester group of the linker reacts with the amine group of the cap protein via NHS ester amine reaction, and the pyridyldithiol group of the linker reacts with the sulfhydryl group of the carrier protein via pyridyldithiol to sulfhydryl reaction.

According to some embodiments, the carrier protein having the plurality of the linkers linked thereon refers to an “activated carrier protein,” whereas the carrier protein without any linker linked thereon refers to an “unconjugated carrier protein.” Also, the molecular weight difference between the activated carrier protein and the unconjugated carrier protein may result from the increase in molecular weight caused by chemical activation. An “average activation degree (AAD)” is used to depict the linkage level for the plurality of the linkers linking to the surface of the carrier protein (i.e., the average number of the linkers binding to each of the carrier protein), which is calculated by the following an equation of.

$$A A D = ( CX - C ) / X$$

in which CX, C, and X are independently representing the molecular weight of the activated carrier protein, the unconjugated carrier protein, and the net weight gain per activation. According to some embodiments of the present disclosure, the range of AAD is between about 1 to 100, for example, the range of AAD may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; preferably, the range of AAD is between about 1 to 50; more preferably, the range of AAD is between about 5 to 25; even more preferably, the range of AAD is between about 10 to 20. According to one specific embodiment of the present disclosure, the range of AAD is about 15.

The value of AAD may vary with the types of the carrier protein. According to some embodiments, the carrier protein is CRM197, and the AAD may range from 10 to 30. In one example, the AAD of CRM197 is 15. In another example, the AAD of CRM197 is between 20 to 25.

Further, the activated carrier protein may further conjugate with the recombinant polypeptide. In the case, a “peptide-protein ratio (PPR)” is used to depict the conjugation level for the plurality of the recombinant polypeptides conjugating to the surface of the activated carrier protein through the linkers (i.e., the average number of the recombinant polypeptides binding to each of the activated carrier protein), which is calculated by an equation of:

$$P P R = \frac{\frac{\sum ( CXPc * Int . c )}{\sum Int . c} - C X}{nX}$$

in which CXPc and Int.c are independently representing the molecular weight and signal intensity of the activated carrier protein (e.g., CRM197) with conjugation number c, whereas CX and nX are independently representing the molecular weight of the activated carrier protein (e.g., CRM197) and the net weight gain per conjugation. In general, PPR is less than or equal to AAD, and PPR in general is between about 1 to 100, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, preferably, the PPR is ranged between about 1 to 50; more preferably, between about 1 to 25; even more preferably, between about 1 to 10. According to some preferred embodiments of the present disclosure, the PPR is about 2, 3, 5, 7, 8, or 10.

The PPR may vary with the types of the carrier protein. According to some embodiments, the carrier protein is CRM197, and the PPR ranges from 5 to 20. In one example, the PPR of CRM197 is 10.

Optionally, after the activated carrier protein is conjugated with the recombinant polypeptide, the conjugate may be further coupled with the cap protein in order to cap the residual free linkers on the surface of the activated carrier protein that are not occupied by the recombinant polypeptides. The cap protein may be conjugated/coupled to the activated carrier protein via the same or similar method described above. The capping step may reduce unwanted chemical reactions occurred on the conjugate, thereby increasing its stability.

The molecular weight of the aforementioned molecules (including the activated carrier protein, the unconjugated carrier protein, the conjugate) may be determined by methods well known in the art, such as gel filtration (also named size exclusion chromatography (SEC)); gradient electrophoresis (e.g., native-polyacrylamide gel electrophoresis (native-PAGE), sodium dodecyl sulfate-PAGE (SDS-PAGE)); mass spectrometry (e.g., matrix-assisted laser desorption ionization-time of flight mass spectrometry (MALDI-TOF MS), MALDI-TOF/TOF MS, matrix assisted laser desorption/ionization-triple quadrupole-tandem mass spectrometry (MALDI-QqQ-MS/MS), surface-assisted laser desorption/ionization-time of flight mass spectrometry (SALDI-TOF MS), surface-enhanced laser desorption/ionization-time of flight mass spectrometry (SELDI-TOF MS), gas chromatography-mass spectrometry (GC-MS), liquid chromatography-mass spectrometry (LC-MS), capillary electrophoresis-mass spectrometry (CE-MS), ion-mobility spectrometry-mass spectrometry (IMS-MS)). According to one working example of the present disclosure, the molecular weight is determined by gel filtration and MALDI-TOF MS.

The present conjugate may be formulated with a pharmaceutically acceptable carrier to form a pharmaceutical composition. A pharmaceutically acceptable carrier is any carrier suitable for in vivo administration. Suitably, the pharmaceutically acceptable carrier is acceptable for oral, nasal or mucosal delivery. The pharmaceutically acceptable carrier may include water, buffered solutions, glucose solutions or bacterial culture fluids. Additional components of the compositions may suitably include excipients such as stabilizers, preservatives, diluents, emulsifiers and lubricants. Examples of pharmaceutically acceptable carriers or diluents include stabilizers such as carbohydrates (e.g., sorbitol, mannitol, starch, sucrose, glucose, dextran), proteins such as albumin or casein, protein-containing agents such as bovine serum or skimmed milk and buffers (e.g., phosphate buffer). Especially when such stabilizers are added to the composition, the composition is suitable for freeze-drying or spray-drying.

Alternatively, the present conjugate may be formulated with an adjuvant to form an immunogenic composition. An “adjuvant” refers to a compound that, when used in combination with a specific immunogen (e.g., the present conjugate) in a formulation, will specifically or non-specifically augment, alter or modify the resultant immune response. Modification of the immune response includes intensification or broadening the specificity of either or both antibody and cellular immune responses. Modification of the immune response can also mean decreasing or suppressing certain antigen-specific immune responses. Examples of adjuvants include an oil emulsion (e.g., complete or incomplete Freund's adjuvant), an oil in water emulsion adjuvants (e.g., the Ribi adjuvant system), syntax adjuvant formulation containing muramyl dipeptide, aluminum salt adjuvant (e.g., aluminium hydroxide, Alhydrogel), polycationic peptide (e.g., polyarginine), oligodeoxynucleotide containing non-methylated cytosine-guanine dinucleotides, human growth hormone, a chemokine (e.g., defensins 1 or 2, RANTES, MIP1-α, MIP-2), a cytokine (e.g., interleukin-1β, -2, -6, -8, -10, or -12; interferon-γ; tumor necrosis factor-α; or granulocyte-monocyte-colony stimulating factor), a muramyl dipeptide variant (e.g., murabutide, threonyl-MDP or muramyl tripeptide), a heat shock protein, Leishmania eukaryotic initiation factor (LeIF), bacterial ADP-ribosylating exotoxins (bAREs), QS21, Quill A, and N-acetylmuramyl-L-alanyl-D-isoglutamyl-L-alanine-2-[1,2-dipalmitoyl-s-glycero-3-(hydroxyp hosphoryloxy)]ethylamide (MTP-PE). An adjuvant may be administered with an antigen or may be administered by itself, either by the same route as that of the antigen or by a different route than that of the antigen. A single adjuvant molecule may have both adjuvant and antigen properties. In one working example, the adjuvant is Alhydrogel.

### 3. Methods for Treating Cancers

Accordingly, it is a further aspect of the present disclosure to provide a method of treating cancers. The method takes advantages of the present conjugate as described in Section 2, in which an effective amount of the present conjugate is administered to a subject having a cancer, so as to suppress or inhibit the growth and/or metastasis of the cancer.

According to some embodiments of the present disclosure, the cancer may be bladder cancer, biliary cancer, bone cancer, brain tumor, breast cancer, cervical cancer, colorectal cancer, esophageal cancer, epidermal carcinoma, gastric cancer, gastrointestinal stromal tumor (GIST), glioma, hematopoietic tumors of lymphoid lineage, hepatic cancer, Kaposi's sarcoma, leukemia, lung cancer, lymphoma, intestinal cancer, melanoma, myeloid leukemia, pancreatic cancer, prostate cancer, retinoblastoma, ovary cancer, renal cell carcinoma, spleen cancer, squamous cell carcinoma, thyroid cancer, and thyroid follicular cancer. In one specific embodiment of the present disclosure, the cancer is breast cancer. Without being bound by theory, the cancer as described herein is an in situ cancer or a metastatic cancer. In one specific embodiment, the cancer is a metastatic cancer.

The effective amount of the present conjugate may vary among different subjects, depending on factors as described above. A therapeutically effective amount may be included in a single dose or multiple doses. In certain embodiments, when multiple doses are administered to a subject, the frequency of administering the multiple doses to the subject is three doses per day, two doses per day, one dose per day, one dose every other day, one dose every third day, one dose per week, one dose every other week, one dose per month, one dose every other month, one dose per season, one dose every half year, or one dose per year. In certain embodiments, the frequency of administering the multiple doses to the subject is one dose per week. In certain embodiments, the frequency of administering the multiple doses to the subject is one dose every other week. In certain embodiments, when multiple doses are administered to a subject, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject. In one specific embodiment, the duration between the first dose and last dose of the multiple doses is about three weeks.

In certain embodiments, a dose (e.g., a single dose, or any dose of multiple doses) described herein includes independently between about 10 ng and 100 μg, inclusive, of the present conjugate, for example, about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 ng, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 81, 82, 83, 84, 85, 86, 87, 88, 89, 90, 91, 92, 93, 94, 95, 96, 97, 98, 99, or 100 μg. In certain embodiments, a dose described herein includes independently between 50 ng and 5 μg, inclusive, of the present conjugate. In one specific embodiment, a dose described herein includes independently 500 ng, inclusive, of the present conjugate.

In the present methods, the present conjugate can be administered by a suitable route as known to those skilled in the art, including oral, intravenous, intraarterial, intracardial, intracutaneous, subcutaneous, transdermal, or intramuscular administration. In general, the most appropriate route of administration will depend upon a variety of factors including the nature of the agent (e.g., its stability in circulation), and/or the condition of the subject (e.g., whether the subject is able to tolerate intravenous administration).

Accordingly, a pharmaceutical composition comprising the present conjugate, and use of the present conjugate in manufacturing a cancer vaccine are encompassed in the scope of the present disclosure.

### EXAMPLES

### Materials and Methods

### 1. Preparation of the Conjugates

Preparation of CRM197 stock solution: To start with, lyophilized CRM197 was dissolved in pre-chilled deionized water; the solvent was exchanged to pre-chilled PBS by performing triple successive rounds of filtration using a 30 kDa MWCO ultra centrifugal filter unit; and the final concentration of the solution was adjusted to 1 mg/ml. All the procedures were carried out at 4° C. or on ice.

Activation by SBAP: Activation of CRM197 by SBAP was carried out with 0.38 mg/ml CRM197 and SBAP under the molar ratio of CRM197:SBAP=1:240, and the pH of the mixture was adjusted to pH=8 with 0.1 M phosphate buffer. The reaction was carried on for 2 hours in the dark, during which the mixture was sufficiently stirred before the reaction was stopped by exchanging the solvent of the mixture to deionized water by performing three successive rounds of filtration using a 30 kDa MWCO ultra centrifugal filter unit. A small aliquot of the mixture was collected for later MALDI-TOF analysis. Then, the pH of the mixture was further adjusted to pH=9 with 0.1M phosphate buffer by performing another three successive rounds of filtration using a 30 kDa MWCO ultra centrifugal filter unit. All the procedures were carried out at 4° C. or on ice.

Peptide conjugation: Peptide conjugation was carried out with 0.15 mg/mI CRM197 under the molar ratio of CRM197:peptide=1:30, 1:60, or 1:90, and the pH of the mixture was adjusted to pH=9 with 0.1M phosphate buffer. The reaction was carried on for 20 hours in the dark, during which the mixture was sufficiently stirred before the reaction was stopped by adding 10 mg L-cysteine directly into the mixture, followed by incubation for 15 minutes. Next, the solvent of the mixture was exchanged to deionized water by performing three successive rounds of filtration using a 30 kDa MWCO ultra centrifugal filter unit. A small aliquot of the mixture was collected for later MALDI-TOF analysis. Afterwards, the solvent of the mixture was further exchanged from deionized water to PBS by performing three successive rounds of filtration using a 30 kDa MWCO ultra centrifugal filter unit. At last, the conjugate was filtered using a 0.22 μm filter unit before lyophilization. All the procedures were carried out at 4° C. or on ice before lyophilization.

### 2. Mass Spectrometry

Properly desalted samples (including Carrier Protein, Carrier:XLnkr, and Vaccine) were mixed with matrix (containing 50% acetonitrile, 0.1% trifluoroacetic acid, and 10 mg/ml sinapinic acid) at the ratio of 1:1 on parafilm before subjected to co-crystalisation on a MALDI-TOF MS sample plate. The samples were then inspected for their m/z value by a MALDI TOF/TOF MS instrument. The raw data were subjected to post-smoothing and background subtracting processes using Savizky-Golay and sensitive nonlinear iterative peak (SNIP) methods with the half window and iteration value at 30 and 190, independently.

### 3. Cell Cultures

Human embryonic kidney 293T (HEK293T), human breast carcinoma cell line MDA-MB-468, and mouse mammary carcinoma cell line 4T1 transduced with green fluorescent protein (GFP) and luciferase (GL-4T1) were maintained in Dulbecco's Modified Eagle's Medium (DMEM) and Roswell Park Memorial Institute 1640 (RPMI1640) medium, respectively, supplemented with 10% fetal bovine serum (FBS), and antibiotics/antimycotics. Cells were grown at 37° C. with a humidified atmosphere of 5% CO₂.

### 4. IL-7RB.ECD Construct

A vector expressing human IL-17RB ectodomain (rhIL-17RB.ECD) was constructed and confirmed by routine laboratory practices. The vector was delivered and expressed in E. coli, and then the resulting recombinant protein was purified for later use.

### 5. Animal Studies

Animal breeding and welfare: Female Balb/c mice about 6-week old were used at starting point of each animal study, which were housed in a climate-controlled room under 12 h/12 h light/dark cycle with food and water ad libitum.

Animal immunization scheme 1: To evaluate the immunogenicity of the present vaccine in mice, female Balb/c mice (6-week old) were arbitrarily divided into two groups: the Carrier (CRM197) group (n=5), and the Vaccine (CRM197-SBAP-hIRIS1 or CRM197-SBAP-hIRIS2) group (n=5). Mice in each group were subjected to blood collection on Day 0, Day 14 and Day 28 during the experiment, and received two doses subcutaneously on Day 1 and Day 15 with 5 μg CRM197 plus 50 μl adjuvant (Alhydrogel) in PBS at 100 μl volume (the Carrier group); or 5 μg CRM197-SBAP-hIRIS1 or CRM197-SBAP-hIRIS2 plus 50 μl adjuvant (Alhydrogel) in PBS at 100 μl volume (the Vaccine group).

Animal immunization scheme 2: To evaluate the immune response induced by the present vaccine against tumors, female Balb/c mice (6-week old) were arbitrarily divided into three groups: the PBS group (n=7), the Carrier (CRM197) group (n=8), the Vaccine (CRM197-SBAP-hIRIS2) group (n=8). Mice in each group were transplanted with tumors (GL-4T1) on Day 0, subjected to blood collection along the experiment, and received five doses subcutaneously on Day 1, Day 7, Day 13, Day 19, and Day 24 with PBS alone (the PBS group); 5 μg CRM197 plus 50 μl adjuvant (Alhydrogel) in PBS at 100 μl volume (the Carrier group); or 5 μg CRM197-SBAP-hIRIS2 plus 50 μl adjuvant (Alhydrogel) in PBS at 100 μl volume (the Vaccine group). The mice were sacrificed at the endpoint of the experiment on Day 34.

Tumor transplantation: One week prior to tumor transplantation, cryopreserved GL-4T1 was revived and grown in complete culture media, and the GFP signal of the GL-4T1 cells was checked by fluorescence microscopy or flow cytometry to ensure the GFP signal was present in the majority (>90%) of the cells. On the day of transplantation, the GL-4T1 cells were trypsinized and washed with PBS before resuspended in Matrigel. The GL-4T1 cells (1×10³ cells at 20 μl volume) were then orthotopically injected into the 4^{th} mammary fat pad of each mouse.

In vivo luciferase signal imaging: Mice were anesthetized with isoflurane to collect the syngeneic tumors and the lungs, and subjected to cardiac puncture to collect the endpoint serum before euthanized by cervix dislocation. The tumors and the lungs were then preserved in ice-cold PBS. Each of the tumors was weighted, and fixed in 10% formalin for later paraffin embedding and sectioning. The lungs were dipped in D-luciferin solution (in PBS) before imaged by an in vivo imaging system. Images of each of the samples were taken for twice consecutively, each for 30 seconds, and then processed by living image software or ImageJ.

Immunohistochemistry: For immunohistochemistry, the sectioned tumor samples were stained with anti-ERK1/2 antibody (1:20 dilution, GeneTex), with counterstained with hematoxylin before being subjected to quantitative analysis.

Serum acquisilion and ELISA: For serum acquisition, the whole blood from the mice was obtained by submandibular vein puncture, and centrifuged at 13,200 RPM for 2 minutes. The supernatant was collected and centrifuged again at 13,200 RPM for 2 minutes to remove the residual blood cells and clots before stored at −20° C. 50 μl of 5 μg/ml purified recombinant human or mouse IL-17RB ectodomain (diluted in PBS) per well was coated in a 96-well ELISA plate at 4° C. overnight. Then, the plate was washed with PBS with 0.1% Tween-20 (PBST) for three times, 5 minutes each time, before blocked with 1% BSA at 37° C. for 1 hour. The plate was washed with PBST for 3 times, 5 minutes each time, and the mouse sera were added into the plate and incubated at 37° C. for another hour. The plate was washed with PBST 3 times, 5 minutes each time, and an anti-mouse IgG-HRP was added into the plate and incubated at 37° C. for 1 hour. Afterwards, the plate was washed with PBST for 3 times, 5 minutes each time, and subjected to 3,3′5,5′-Tetramethylbenzidine (TMB) color reaction for 2 minutes before stopped by adding 1M HCl. At last, the absorbance of the plate was measured for optical density (OD) 450 nm by a microplate reader.

### 6. Statistics

Independent Student's t-tests to compare between two groups were performed, and the experimental data were expressed as mean standard deviation (mean+SD). The experiments were repeated at least three times. P<0.05 was considered as statistically significant.

### Example 1 Production of Vaccine

To evaluate the possibility of utilizing the IRIS epitope as a cancer vaccine, various conjugates independently containing a recombinant polypeptide having one or more copies of the synthesized murine or human IRIS sequence were constructed in accordance with procedures described in the “Materials and Methods” section. The recombinant polypeptides were mIRIS1 (containing one copy of the synthesized murine IRIS sequence), hIRIS1 (containing one copy of the synthesized human IRIS sequence), mIRIS2 (containing two copies of the synthesized murine IRIS sequence), hlRIS2 (containing two copies of the synthesized human IRIS sequence), mIRISn (containing n copies of the synthesized murine IRIS sequence), and hIRISn (containing n copies of the synthesized human IRIS sequence); SEQ ID NOs: 3 to 12). Two auxiliary glycine residues were added to C-terminal of each copy of the murine or human IRIS sequence, and the C-terminal of the recombinant polypeptide was further fused with a conjugation site of choice. In addition, the N-terminal of the recombinant polypeptide was acetylated to prevent unwanted reactions occurred between the recombinant polypeptide and the linker used in the studies. As an example, the conjugate containing a recombinant polypeptide having two copies of the synthesized murine or human IRIS sequence (n=2; mIRIS2 or hIRIS2) (SEQ ID NOs: 5 and 6) was used to produce a cancer vaccine in the following studies.

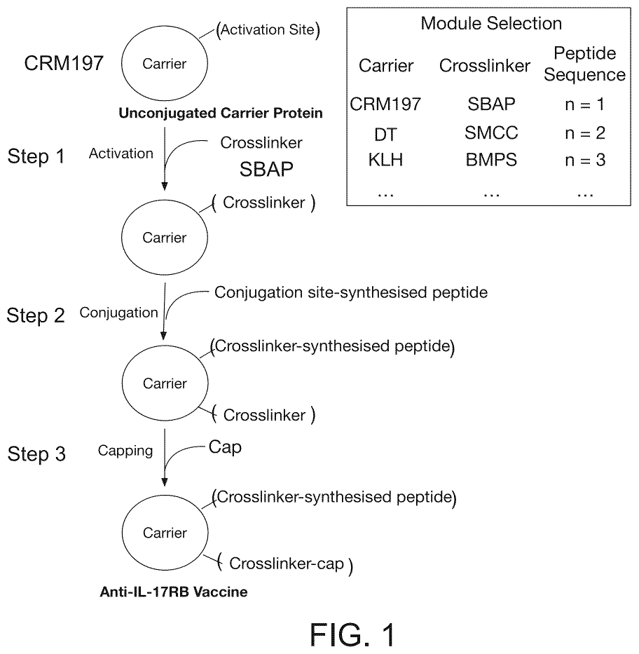

The cancer vaccine is produced in accordance with procedures depicted in FIG. 1. Briefly, an unconjugated carrier protein CRM197 with multiple active sites (about 40 primary amine groups) on its surface was activated with a compatible linker SBAP (Step 1, Activation). Empirically, about 20 to 25 out of the 40 primary amine groups on CRM197 may be activated. Then, the activated carrier protein was conjugated with the recombinant polypeptide fused with a suitable conjugation site as described above, thereby producing the conjugate (Step 2, Conjugation). The unconjugated sites on CRM197 were capped to produce an anti-IL-17RB vaccine (Step 3, Capping).

### Example 2 Chemical Characterization of the Vaccine

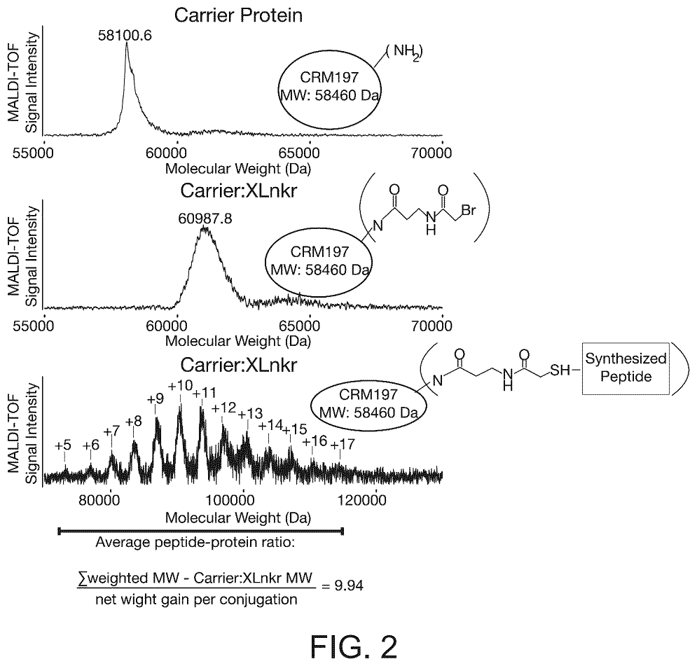

The resultant vaccine of Example 1 was qualitatively and quantitatively characterized by mass spectrometry (MS) in this Example. In particular, the vaccine was first analyzed by MS, and the MS data were used to calculate the peptide-protein ratio (PPR), a general quantitative characteristics of chemical conjugates. The molecular weight of the unconjugated CRM197 (i.e., Carrier Protein), CRM197-SBAP (i.e., Carrier:XLnkr), and CRM197-SBAP-hlRIS2 (i.e., Vaccine) was determined by MALDI-TOF, and results are provided in FIG. 2. As compared with the unconjugated CRM197, a visible molecular weight shift was found in CRM197-SBAP. The difference between these two proteins was used to calculate the average activation degree (AAD) using equation (1), which reflects the average linker number attached to each CRM197:

$$A A D = ( C X - C ) / X \qquad ( 1 )$$

in which CX and C are independently the molecular weight of CRM197-SBAP and CRM197, and X is the net weight gain per activation. The AAD of CRM197-SBAP (i.e., Carrier:XLnkr) was estimated to be 15.04 (about 15), which meant each CRM197 gained 15.04 SBAP (about SBAP; the k value) molecules in average.

Further, a wide distribution of peaks was found in CRM197-SBAP-hIRIS2, which reflected the number (ranging from 5 to 17, calculated by the following PPR formula) of the recombinant polypeptides hIRIS2 conjugated to CRM197-SBAP, and the signal intensity of each peak reflected the amount of the CRM197-SBAP-hIRIS2 with the indicated number of hIRIS2 conjugated thereto. According to the MS data, the PPR was calculated by equation (2),

$$PPR = \frac{\frac{\sum ( CXPc * Int . c )}{\sum Int . c} - C X}{nX} \qquad ( 2 )$$

in which CXPc and Int.c are respectively the molecular weight and signal intensity of CRM197-SBAP-hIRIS2 with the indicated conjugation number c; CX and nX are respectively the molecular weight of CRM197-SBAP and net weight gain per conjugation. The PPR of CRM197-SBAP-hIRIS2 (i.e., Vaccine) was estimated to be 9.94 (about 10), which meant each CRM197-SBAP gained 9.94 hIRIS2 (about 10 hIRIS2) molecules in average.

### Example 3 Effects of the Vaccine on Induction of Mouse Humoral Immune Responses

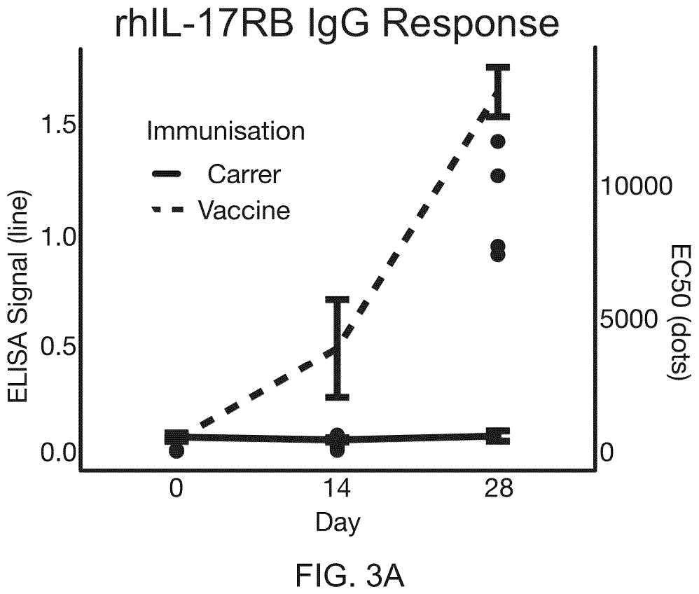

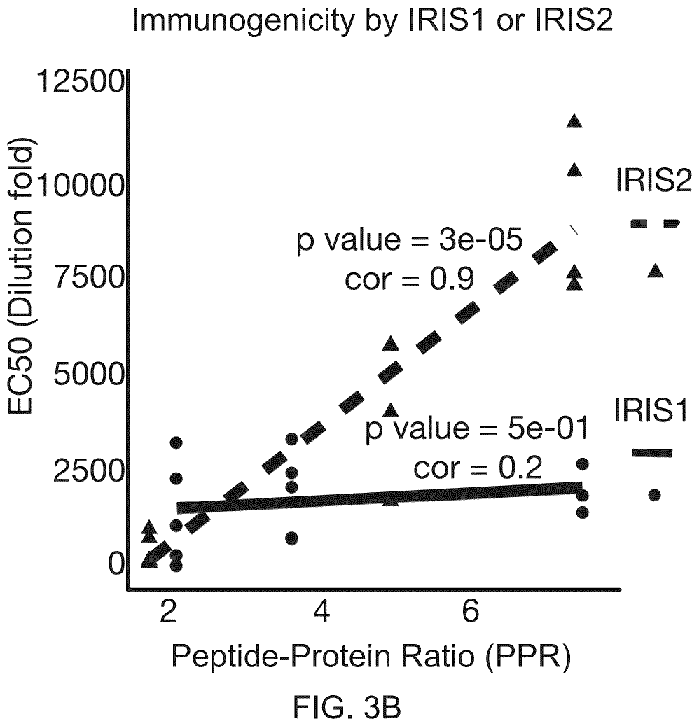

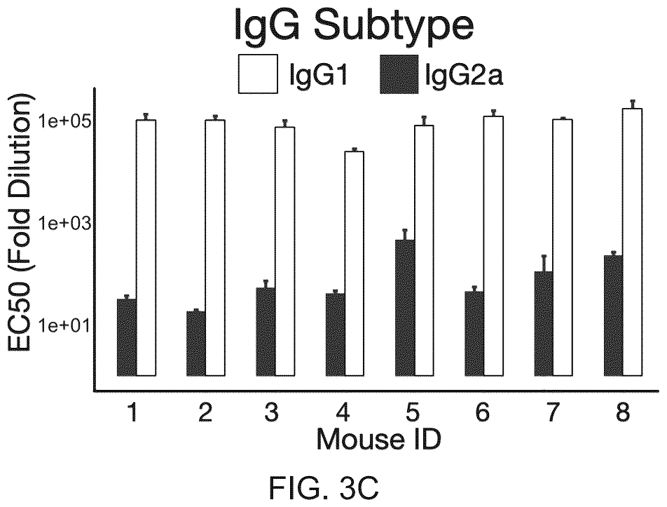

In this example, the in vivo immunogenicity of the present vaccine was investigated. To this end, Balb/c mice were immunized with the vaccine CRM197-SBAP-hIRIS2 (n=5; the Vaccine group), or the control CRM197 (n=5; the Carrier group). For each group, a total of two doses of the indicated treatments were given (5 μg/dose) along with Alhydrogel adjuvant (50 μg/dose), and the two doses were given on Days 1 and 15, respectively. Mouse sera were collected prior to vaccination (Day 0), during vaccination (Day 14), and also subsequent to vaccination (Day 28), and the antibodies therein against the purified recombinant human IL-17RB ectodomain (rhIL-17RB.ECD) were examined by ELISA. According to the data provided in FIG. 3A, the sera collected on Day 28 from the mice immunized with the vaccine exhibited a prominent reactivity against rhIL-17RB.ECD, indicating strong immune responses were induced in the mice, whereas no detectable reactivity was found in the sera from the mice immunized with the control CRM197. Taken together, these data indicates that the vaccine, rather than CRM197, is capable of inducing humoral immune responses against rhIL-17RB.ECD within the vaccinated mice.

Further, the immunogenicity of the vaccine conjugated with hRIS1 or hIRIS2 (i.e., CRM197-SBAP-hIRIS1 or CRM197-SBAP-hIRIS2), as well as the vaccines of various PPRs were investigated in the present study.

To this purpose, the vaccines of various PPRs were prepared, in which the PPRs of the CRM197-SBAP-hIRIS1 were 2.09, 3.63 and 7.51, and the PPRs of the CRM197-SBAP-hIRIS2 were 1.73, 4.95 and 7.40, respectively. The results were provided in FIG. 3B. It was found that the mice (n=3-5 for each group) immunized with the CRM197-SBAP-hIRIS1 exhibited a weak immune response (i.e., lower correlation between the EC50 and the PPRs). By contrast, the mice immunized with the CRM197-SBAP-hIRIS2 exhibited a better immune response (i.e., linear correlation between the EC50 and the PPRs). Further, the dominant IgG subtype was verified by ELISA, and the results clearly indicated that dominant IgG subtype was IgG1 rather than IgG2a (FIG. 3C), suggesting the T_{H}2 immune response tends to be triggered under the context of immunization. Collectively, these data evidences that the vaccine CRM197-SBAP-hIRIS2 has a prominent immunogenicity, especially for which at higher PPRs, and the immune response under such immunization condition is mainly the DH2-type.

### Example 4 the Present Vaccine Suppressed Tumor Growth and Metastasis in a Syngeneic Mouse Model

The ability of the present vaccine to suppress tumor growth and metastasis in a syngeneic mouse tumor model was investigated in the present example. First, 1×10³ 4T1-GFP-luc cells were orthotopically transplanted into the 4^{th} mammary fat pad of Balb/c mice on Day 0 before the mice were primed with PBS (n=7; the PBS group), CRM197 (n=8; the Carrier group), or CRM197-SBAP-mlRIS2 (n=8; the Vaccine group). One week later, the luciferase signal from the transplanted cells were examined by an in vivo imaging system (IVIS) to check the viability of the cells. The vaccination scheme was provided in “Materials and Methods”, a total of five doses of the indicated treatments were given, and the five doses were given on Day 1, Day 7, Day 13, Day 19, and Day 24, respectively. On Day 34, the mice were sacrificed, and the tumors and the lungs were harvested for further analysis.

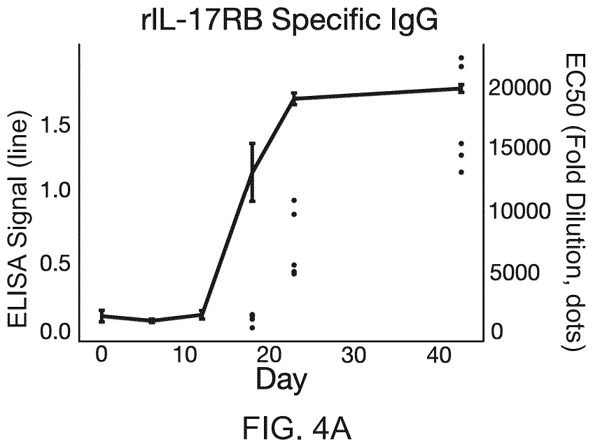

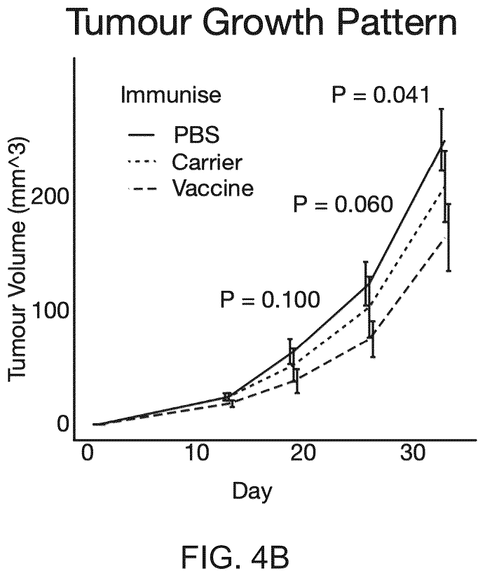

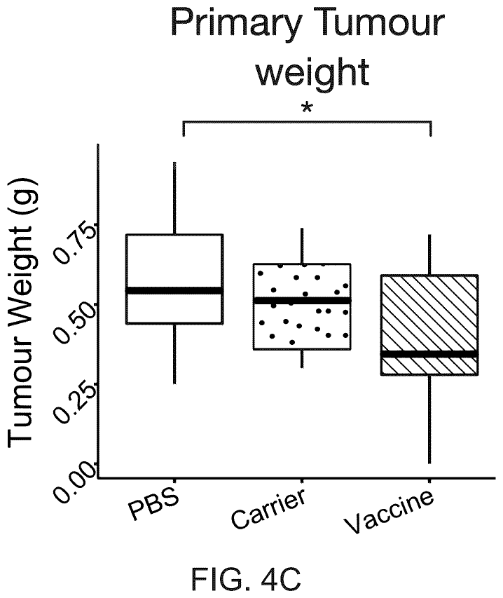

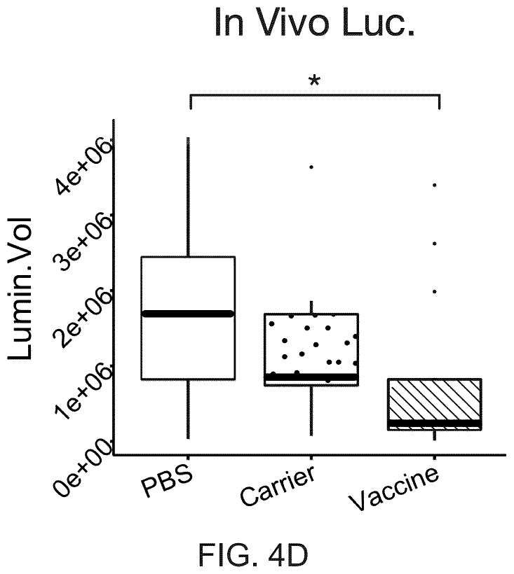

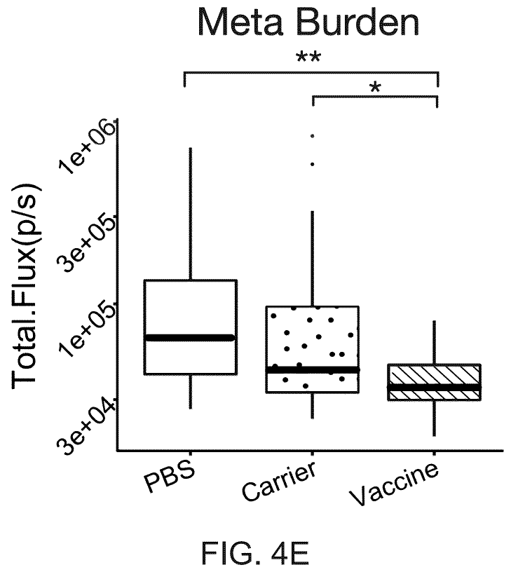

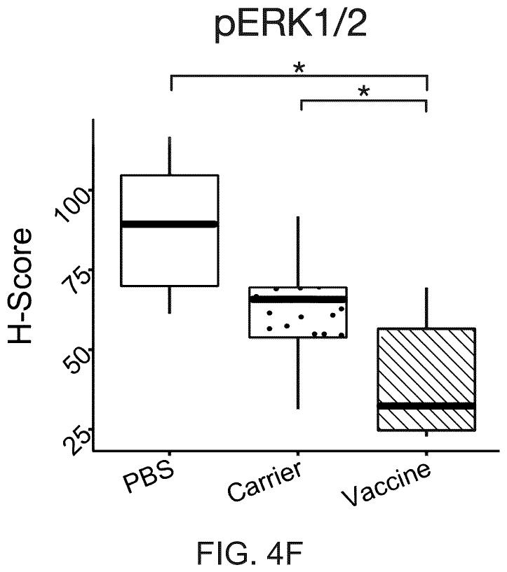

It was found that the mice immunized with the vaccine successfully produced a large number of IgGs against IL-17RB, in particular, after Day 10 of vaccination (FIG. 4A). In addition, on Day 20 after transplantation, a considerable difference in the primary tumor size was found between the PBS and the Vaccine groups (FIG. 4B), the time approximately corresponded to the time when IgG response emerged (FIG. 4A). The difference in the primary tumor size between the two groups became more and more evident over time and reached statistical significance (p<0.05) at the end of the study (FIG. 4B). The same phenomenon was also observed in the primary tumor weight at the endpoint of the study; a clear decrease in the primary tumor weight was found in the Vaccine group as compared to that in the PBS group (FIG. 4C). Moreover, the intensity of the luminescence emitted from the primary tumors in the Vaccine group was far less than that in both of the control groups on Day 13 (FIG. 4D). Taken together, these data evidenced that the present vaccine has the ability to trigger the immune response against IL-17RB, which in turn helps suppress primary tumor outgrowth. The present vaccine is validated to be a promising treatment in treating cancers.

The effects of the present vaccine on suppressing tumor migration were also investigated. At the endpoint of the experiment, the lung tissues were harvested and subjected to luminescence detection. Large, multi-foci metastatic clumps were found in both of the control groups (i.e., the PBS and the Carrier groups), whereas only small, singular metastatic spots were found in the Vaccine group (data not shown). Further, the metastatic tumors were quantified by detection of the luminescence intensity in the lungs, and it was found that the luminescence intensity in the Vaccine group was significantly decreased as compared to that in both of the control groups (FIG. 4E). These data remarkably indicated that the disease progression and dissemination has been compromised by treating with the present vaccine.

The tumor metastasis was further evaluated with another analytical method, a risk difference hypothesis testing. The results are summarized Table 1. The distant metastasis risk in the Vaccine group was found to be nearly 50% drop as compared to that in both of the control groups (Table 1). Collectively, these data strongly evidenced that the present vaccine may suppress distal metastasis of the cancer.

TABLE 1
The distant metastasis risk of the tumors
in the indicated treatment groups

| | Metastasis | | |
|---|---|---|---|
| Immunization | True | False | Risk |
| PBS | 5 | 2 | 71% |
| Carrier | 6 | 2 | 75% |
| Vaccine | 2 | 6 | 25% |

The underlying mechanism fortumorigenesis, in particular, the contribution of IL-17RB in tumorigenesis via ERK1/2 dependent cell activation was also examined. To this purpose, primary tumor sections stained with IHC were examined for the presence of the activation form of ERK1/2 (i.e., the phosphorylated ERK1/2, pERK1/2), and results are provided in FIG. 4F. The expression level of the pERK1/2 in the Vaccine group was remarkably lower than that in both of the control groups. Thus, these data suggested that the present vaccine contributes to suppression of tumor progression through inhibition of ERK1/2 pathway.

In sum, the present invention provides a promising cancer vaccine that may effectively suppress tumor growth and metastasis.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A recombinant polypeptide, comprising 1 to 20 copies of an interleukin-17 receptor B (IL-17RB) inactivation site (IRIS) sequence having an amino acid sequence at least 85% identical to SEQ ID NO: 1 or SEQ ID NO: 2, wherein when more than one copy of the IRIS sequence is present in the recombinant polypeptide, each copy of the IRIS sequence is serially connected to each other.

2. The recombinant polypeptide of claim 1, wherein the IRIS sequence has the amino acid sequence 100% identical to SEQ ID NO: 1 or SEQ ID NO: 2.

3. The recombinant polypeptide of claim 1, wherein
the N-terminus of the recombinant polypeptide is acetylated, formylated, methylated, carbamylated, pegylated, phosphorylated, or glycosylated, and/or
the C-terminus of the recombinant polypeptide is amidated, glypiated, biotinylated, or glycosylated.

4. A conjugate, comprising
a carrier protein;
a plurality of the recombinant polypeptides of claim 1; and
a plurality of linkers for linking the plurality of the recombinant polypeptides to the carrier protein;
wherein,
each of the linkers is linked to each of the recombinant polypeptides at one end and the carrier protein at the other end independently through NHS ester amine reaction, thio-succinimide reaction, pyridyldithiol to sulfhydryl reaction, bromoacetyl to sulfhydryl reaction, or iodoacetyl to sulfhydryl reaction.

5. The conjugate of claim 4, wherein the carrier protein is edema factor (EF) of Bacillus anthracis, lethal factor (LF) of Bacillus anthracis, bovine serum albumin (BSA), CRM9, CRM45, CRM102, CRM103, CRM107, CRM176, CRM197, CRM228, diphtheria toxoid, heat-labile enterotoxin (LT) of Escherichia coli, heat-stable enterotoxin (ST) of Escherichia coli, human serum albumin, keyhole limpet hemocyanin (KLH), ovalbumin, pertussis toxoid, pneumococcal adhesin protein A (PsaA), pneumococcal surface protein A (PspA), pneumolysin, porins, exotoxin A from Pseudomonas aeruginosa, tuberculin, tetanus toxoid, or transferrin binding protein.

6. The conjugate of claim 5, wherein the carrier protein is CRM197.

7. The conjugate of claim 4, wherein the linker is succinimidyl 3-(bromoacetamido) propionate (SBAP), succinimidyl 4-(N-maleimido methyl) cyclohexane-1-carboxylate (SMCC), or N-β-maleimidopropyl-oxysuccinimide ester (BMPS).

8. The conjugate of claim 4, wherein
the N-terminus of the recombinant polypeptide is acetylated, formylated, methylated, carbamylated, pegylated, phosphorylated, or glycosylated, and/or
the C-terminus of the recombinant polypeptide is amidated, glypiated, biotinylated, or glycosylated.

9. A method for treating a cancer in a subject, comprising administering to the subject an effective amount of the conjugate of claim 4.

10. The method of claim 9, wherein the cancer is any one of bladder cancer, biliary cancer, bone cancer, brain tumor, breast cancer, cervical cancer, colorectal cancer, esophageal cancer, epidermal carcinoma, gastric cancer, gastrointestinal stromal tumor (GIST), glioma, hematopoietic tumors of lymphoid lineage, hepatic cancer, Kaposi's sarcoma, leukemia, lung cancer, lymphoma, intestinal cancer, melanoma, myeloid leukemia, pancreatic cancer, prostate cancer, retinoblastoma, ovary cancer, renal cell carcinoma, spleen cancer, squamous cell carcinoma, thyroid cancer, or thyroid follicular cancer.

11. The method of claim 10, wherein the subject has the breast cancer.

12. The method of claim 9, wherein the cancer is a metastatic cancer.

13. The method of claim 9, wherein the subject is a human.


SEQUENCE LISTING (13 sequences)

SEQ ID NO: 1 - protein, 13 aa, Artificial Sequence
  Synthetic
VQHTLTPGDLRDL

SEQ ID NO: 2 - protein, 13 aa, Artificial Sequence
  Synthetic
LQHDLIPGDLRDL

SEQ ID NO: 3 - protein, 15 aa, Artificial Sequence
  Synthetic
VQHTLTPGDLRDLGG

SEQ ID NO: 4 - protein, 15 aa, Artificial Sequence
  Synthetic
LQHDLIPGDLRDLGG

SEQ ID NO: 5 - protein, 30 aa, Artificial Sequence
  Synthetic
VQHTLTPGDLRDLGGVQHTLTPGDLRDLGG

SEQ ID NO: 6 - protein, 30 aa, Artificial Sequence
  Synthetic
LQHDLIPGDLRDLGGLQHDLIPGDLRDLGG

SEQ ID NO: 7 - protein, 75 aa, Artificial Sequence
  Synthetic
VQHTLTPGDLRDLGGVQHTLTPGDLRDLGGVQHTLTPGDLRDLGGVQHTLTPGDLRDLGGVQHTLTPGDLRDLGG

SEQ ID NO: 8 - protein, 75 aa, Artificial Sequence
  Synthetic
LQHDLIPGDLRDLGGLQHDLIPGDLRDLGGLQHDLIPGDLRDLGGLQHDLIPGDLRDLGGLQHDLIPGDLRDLGG

SEQ ID NO: 9 - protein, 150 aa, Artificial Sequence
  Synthetic
VQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGG

SEQ ID NO: 10 - protein, 150 aa, Artificial Sequence
  Synthetic
LQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGG

SEQ ID NO: 11 - protein, 300 aa, Artificial Sequence
  Synthetic
VQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGGVQHTLTPGDLRDLGG

SEQ ID NO: 12 - protein, 300 aa, Artificial Sequence
  Synthetic
LQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGGLQHDLIPGDLRDLGG

SEQ ID NO: 13 - protein, 536 aa, Artificial Sequence
  Synthetic
MGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS